(19) United States
(12) Patent Application Publication
(10) Pub. No.: US 2015/0178461 A1
(43) Pub. Date: Jun. 25, 2015

(54) GROUP POSTURE HEALTH RISK MANAGEMENT

(71) Applicant: INTERNATIONAL BUSINESS MACHINES CORPORATION, Armonk, NY (US)

(72) Inventors: James R. KOZLOSKI, New Fairfield, CT (US); Timothy M. LYNAR, Kew (AU); Suraj PANDEY, Parkville (AU); John M. WAGNER, Plainville, CT (US)

(73) Assignee: INTERNATIONAL BUSINESS MACHINES CORPORATION, Armonk, NY (US)

(21) Appl. No.: 14/133,790

(22) Filed: Dec. 19, 2013

(51) Int. Cl. G06F 19/00; A61B 5/11; G06Q 10/10

(52) U.S. Cl. CPC G06F 19/3431; G06Q 10/1095; G06Q 40/08; A61B 5/1116

(57) ABSTRACT

The seated posture of a subject person and a group of subject persons over a long term is analyzed to determine long-term health risk factors. Responses to the determined long-term health risk factors include posture improvement messages and group activity recommendations to reduce the long-term health risk factors as well as adjustments to reserves and premiums insuring costs associated with the long-term risks.

### BACKGROUND

This disclosure broadly relates to the field of information processing systems, and more particularly to the field of processing information related to the posture of one or more subject persons and management of associated health risk factors.

Prolonged sitting has been determined to be a risk factor in mortality. In a publication by the American Medical Association entitled “Sitting Time and All-Cause Mortality Risk in 222,497 Australian Adults” by Hidde P. van der Ploeg, PhD; Tien Chev, MAppStats; Rosemary J. Koala, PhD; Emily Banks, MBBS, PhD; and Adrian Bauman, MBBS, PhD, ARCH INTERN MED/VOL 172 (NO. 6), Mar. 26, 2012, it was concluded that prolonged sitting is a risk factor for all-cause mortality independent of physical act and other health related factors, Said publication is hereby incorporated by reference in its entirety. Prolonged sitting was determined to decrease life expectancy independent of health factors such as age, sex, weight, physical activity or health status. While the increase in mortality may vary between health factors, mortality was nevertheless increased in all health factors evaluated. It was found that greater absolute MO risk existed in individuals with existing cardiovascular disease, diabetes, overweight or obesity, yet mortality increase due to prolonged sitting was nevertheless observed in those who engaged in physical activity in excess of five hours per week. The report also suggests that prolonged sitting may adversely affect type 2 diabetes mellitus and cardiovascular mortality and that the public could benefit. from public health programs that focused on reduci rig time.

Individual posture monitoring devices and systems monitor the posture of a subject person in their environment. Monitoring approaches include video monitoring, incorporating sensors on the body or in clothing, incorporating sensors into devices such as desks and chairs, and context awareness that accounts for activities of the person as part of the posture monitoring process. Such monitoring devices and systems may be found in US Patent Application 2007/0149360, entitled “Device for Monitoring a User's Posture”, by Chandrasekhar Narayanaswami and assigned to International Business Machines Corporation, Jun. 28, 2007, and “Smart monitoring of worker posture in an office environment” by Steven Haveman and Gijs Kani (remainder of publication information needed). Said publications are hereby incorporated by reference their entirety. Such posture monitoring devices and systems are able to determine a number of elements of a subject person's posture.

Organizations involved in the welfare of groups of individuals can benefit from reducing the morality rate associated with the group. Reducing an all-cause mortality is most beneficial as the mortality is reduced across all members of the group, independent of age, sex, health condition or physical activity, rather than individual segments of the group.

### SUMMARY

Briefly, in an aspect of the disclosure, long-term group posture analytics are accumulated and processed in order to encourage particular corrective group activities, determine risk factors, and manage any associated health or mortality risk. In another aspect, correctives activities for an individual subject person may be proposed.

An aspect of the disclosure includes a method comprising receiving a seated signal from a device monitoring postures of a subject person, accumulating the seated signal over duration, and generating a health risk factor based upon the accumulating. Furthermore, the method receives a plurality of seated signals from a plurality of devices monitoring postures of a plurality of subject persons, the accumulating further accumulates the plurality of seated signals, and the generating further generates a group health risk factor for the plurality of subject persons based on the accumulating. The method further includes selecting a group activity from a plurality of group activities for the plurality of subject persons based upon the group health risk factor wherein the plurality of group activities includes a seated meeting forum and an unseated meeting forum and the method selects between the seated meeting forum and the unseated meeting forum based upon the group health risk factor. The method further includes adjusting an insurance premium associated with the plurality of subject persons based upon the group health risk factor and a participation in the group activity. The method further wherein the plurality of devices includes a computer system having a calendar application for at least one of the plurality of subject persons and the method further comprises generating the at least one of the plurality of seated signals based upon the calendar application.

Another aspect of the disclosure includes a system comprising: a plurality of posture monitors adapted to analyze a posture of each of a plurality of subject persons and generate a plurality of seated signals based on the analysis; a plurality of posture accumulators adapted to accumulate the plurality of seated signals; a plurality of individual characteristic databases including individual characteristic information on each of the plurality of subject persons; an actuarial database having information relating each of the plurality of posture accumulators to of each of the individual characteristic databases and adapted to be used in generating a plurality of health risk factors; and a long-term health risk factor generator coupled to the actuarial database, the plurality of individual characteristic databases and the plurality of posture accumulators, the long-term health risk factor generator adapted to calculate the plurality of health risk factors and generate a group health risk factor signal. The system further comprising a group activity selector coupled to the long-term health risk factor generator and adapted to select a group activity from a plurality of group activities based upon the group health risk factor signal. The system further comprising a group insurance calculator coupled to the long-term health risk factor generator and adapted to determine at least one of an insurance reserve and an insurance premium based upon the group health risk factor signal.

Another aspect of the disclosure includes a non-transitory computer program comprising a computer readable storage medium having computer readable program code embodied therewith, the computer readable program code configured to: receiving a seated signal from a device monitoring a posture of a subject person; accumulating the seated signal over a duration; and generating a health risk factor based upon the accumulating. The receiving further receives a plurality of seated signals from a plurality of devices monitoring postures of a plurality of subject persons; the accumulating further accumulates the plurality of seated signals; and the generating further generates a group health risk factor for the plurality of subject persons based on the accumulating.

### BRIEF DESCRIPTION OF THE DRAWINGS

The accompanying figures where like reference numerals refer to identical or functionally similar elements throughout the separate views, and which together with the detailed description below are incorporated in and form part of the specification, serve to further illustrate various embodiments and to explain various principles and advantages all in accordance with the present disclosure, in which:

FIG. 1 illustrates an example of a block diagram of a system for group posture health risk management;

FIG. 2 illustrates an example of an individual posture monitor for monitoring a posture of a subject person;

FIG. 3 illustrates an example graph showing deaths per thousand person-years versus hours per day of sitting;

FIG. 4 illustrates a table showing an association between sitting and all-cause mortality among Australian adult forty five years or older; and

FIG. 5 illustrates a flow diagram example of a process for determining health risk factor based upon sitting time and associated responses.

### DETAILED DESCRIPTION

In the following discussion, details are provided to help thoroughly understand the present disclosure. However, it is apparent to those of ordinary skill in the art that even though there may be no such details, the understanding of the present disclosure would not be influenced. In addition, it should be further appreciated that any specific terms or applications used herein are only for the convenience of description, and thus the present disclosure should not be limited to only use in any specific terms or applications represented and/or implied by such terms.

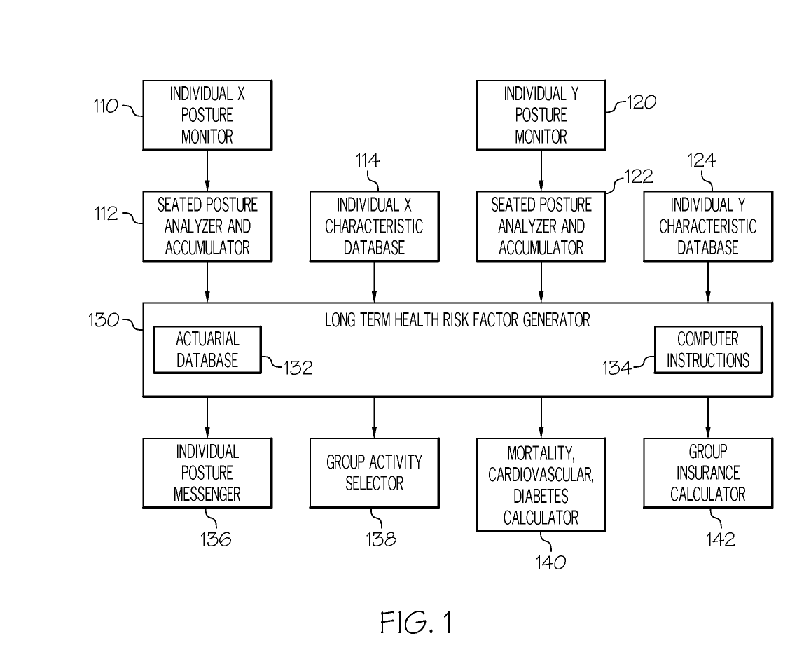

FIG. 1 illustrates an example of a block diagram of a system for group posture health risk management. Individual posture monitor 110 monitors the posture of an individual, in this case individual person “X”. The posture monitor may be a device or context aware posture recognition system that includes video monitoring, incorporates sensors on the body or in clothing, incorporates sensors into devices such as desks and chairs, and may include contextual awareness of activities of the person being monitored. The scope of the disclosure includes all types of posture monitoring and is not limited to the examples of devices and systems for posture monitoring that are included herein. A second individual posture monitor 120 is for monitoring the posture of a second individual person “Y”. The posture monitor may be identical to posture monitor 110 or may be a different posture monitor. Furthermore, the posture of individual X may, at times, be monitored by posture monitor 120. For example if posture monitor 120 includes a video camera system at the office of individual Y, and individual X is visiting with Y and within view of the video camera system, then the camera image could monitor the posture of both X and Y and use facial recognition of other identification approach to identify X and Y. Based on the camera image, signals indicative of the posture of both X and Y may be generated. Posture signals associated with subject person X are then analyzed by seated posture analyzer and accumulator 112. There may be numerous posture monitors in a system. For example, posture monitors may be distributed throughout an office building or other installations and include video monitors, sensors in furniture, and body worn sensors. Any posture monitor may have the capability to generate a posture signal associated with a subject person.

The posture signals for subject person X may be received from any posture monitor 110 or 120. The posture signals are then analyzed to determine if the subject person has a seated posture. Determination of a seated posture is known to those familiar with the art, and the disclosure is not limited to a particular approach for determining a seated posture. In one example, a weight sensor in the seat of a chair may be used to determine that the subject person is in the chair and a threshold amount of weight is supported by the seat of the chair. The threshold weight may be a minimum weight such as twenty pounds or may be based upon the weight of the subject person, such as fifty percent of the weight of the subject person. Such a signal from the weight sensor in the seat would indicate that the subject person has a seated posture. A video system may determine the subject person has a seated posture by determining the subject person is occupying a chair. In another example, a body sensor system may determine an angle between the subject person's torso and thigh indicates a seated position. The seated posture accumulator for each subject person accumulates long-term data and is able to make the data available for analysis over variable long-term intervals such as a week, a month, a year or other interval.

In the absence of sensors, the seated posture may be determined contextually. For example, if the subject person is known to have an hour commute to an office, then it may be reasonable to accumulate the commute time as a seated time. On the other hand if the subject person's calendar shows the subject person is scheduled to play a volleyball game then the duration of the volleyball game may not be accumulated as seated posture time. In another example, audio analysis, social networking analysis or other communications activities (e.g., comments and/or complaints) could be performed on the subject person's communications following their unmonitored activity in order to provide information on their past to help determine seated time. Similarly if the calendar of a subject person shows a time in a meeting room configured for unseated activities such as a standup meeting, that is a meeting where participants are standing up and not seated, then the duration of the meeting may not be accumulated as a seated posture. For times when sensor data or contextual data is not available, then predetermined assumptions may be used to accumulate seated posture. For example, it may be assumed that the subject persons is lying down asleep for thirty percent of the time, and seated for a certain percent of the remaining time that sensor and/or contextual seated signals are not available. The certain percentage may be an arbitrary number such as fifty percent, or based on a survey of the subject person, or extracted from social media or other databases associated with the subject person.

Individual characteristic database 114 includes individual characteristic information related to subject person X, and individual characteristic database 124 includes characteristics related to subject person Y. The database may include sex, age, education level, marital status, location residence, body mass index, smoking status, health status, serological analysis, genetic analysis, and physical activity of the subject person. The individual characteristic database may include other information facilitating identification of the individual, such as photographic images of the person, voice identification of the person, fingerprint identification information, identification of devices carried by the individual such as identification signals embedded in Bluetooth, WiFi, Zigbee, and cell phone devices. While individual posture monitors, seated posture analyzers and accumulators, and individual characteristic databases are shown for two individuals in this example, in other examples, there may be a multiplicity of individual posture monitors, seated posture analyzers and accumulators, and individual characteristic databases to support larger groups.

The accumulated seated posture signals and individual characteristics associated with the subject persons are then processed by long-term health risk factor generator 130. Long-term health risk factor generator generates a health risk factor for each subject individual based upon the accumulated seated signal and other individual characteristics that may be included in the individual characteristic database. The individual health risk factor may be calculated from information in an actuarial table 132 that generates a mortality risk factor based on the individual's characteristic database and accumulated seated posture. The calculation may be done by a computer (not shown) having computer instructions 134 for performing the calculation and/other steps included in this description. The individual health risk factor may then be used to send messages from individual posture messenger 136 for generating a long-term posture modification signal for the subject person based upon the health risk factor. A benefit of the posture modification signal is to modify an amount of time the subject person is seated over a period of time. The long-term posture modification signal may be sent to the individual to encourage corrective actions intended to reduce the determined risk. The corrective actions may include encouraging less time seated and may be tailored to the individual. For example a person who engages in physical activity over four hours a week may be encouraged to engage in activities that reduce their seated time if their long-term average seating time exceeds eleven hours per day, while an overweight person may be encouraged to engage in activities that reduce their seated time if their long-term average seating time exceeds eight hours per day. Note that in another example, individual posture messages may also be based on other posture information included in the posture signal not directly related to a seated or an unseated posture. Such posture messages may encourage other behaviors beneficial to the individual while sitting, such messages may include sitting straighter, changing positions of arms or hands, or even increasing levels of fidgeting to improve components of the health of the person.

Group analytics may also be performed on a number of selected persons who are members of a group in order to determine a long-term health risk factor for the group. Depending upon the risk to be calculated, the calculator 140 may include mortality, cardiovascular and/or diabetes risk or other risks found to be related to the seated posture of a subject person, or a group of subject persons. The calculated risk may be in any of a number of forms including a printed report, a rendering on a computer screen, or a signal to another process or machine for further processing. For example, the signal may be sent to a group activity selector 138 such as a meeting planner process. If a request is received from a meeting planning process for a designated group of individuals, then the health risk factor for the designated group of individuals can be calculated to determine if a meeting location facilitating a seated meeting should be recommended of if a meeting location facilitating a non-seated meeting should be recommended, particularly if the non-seated meeting would help reduce the health risk factor of those involved in the meeting. For example a group of individuals who engage in physical activity between one hundred and fifty and three hundred minutes a week may not benefit as much from a non-seated meeting as a group of overweight individuals who are seated an average of over eleven hours per day. Thus, group activity selector 138 selects from a plurality of group activities including a seated meeting forum and an unseated meeting forum based upon the group health risk factor.

The group health risk factor may also be used for insurance purposes. For example, reserves held for health and/or life insurance may be adjusted based upon the results of calculator 140. This applies whether the group is self-insured or of uses a separate insurance company or entity to manage the insurance of the risk associated with the mortality, and other health issues calculated based upon the seated posture of the group. Monitory reserves are calculated based upon the risk factor to insure against negative consequences of the risk. For example a group at a high risk for cardiovascular disease may require a certain monitory reserve to insure against medical expenses associated with the disease. Maintaining the monitory reserve may require a periodic insurance premium. The disclosure may be used to help provide a more accurate estimate of the risk associated with the group and thus a more accurate determination of the monitory reserve and periodic premium to maintain the reserve. Furthermore, the group activity selector 138 may promote behavior that reduces the health factor risk associated with the group, and the posture monitors 110 and 120 may be used to determine that the health factor risk has been indeed reduced. This, in turn, may reduce the monitory reserve and insurance premiums required to insure against the health risk. A similar analysis applies to group mortality and life insurance reserves and premiums encountered by employers of the various groups and/or insurance companies insuring the various groups.

From a system perspective, FIG. 1 shows a system comprising: a plurality of posture monitors 110, 120 adapted to analyze a posture of each of a plurality of subject persons and generate a plurality of seated signals based on the analysis; a plurality of posture accumulators 112, 122 adapted to accumulate the plurality of seated signals; a plurality of individual characteristic databases 114, 124 including individual characteristic information on each of the plurality of subject persons; an actuarial database 132 having information relating each of the plurality of posture accumulators to of each of the individual characteristic databases and adapted to be used in generating a plurality of health risk factors; and a long-term health risk factor generator 130 coupled to the actuarial database, the plurality of individual characteristic databases and the plurality of posture accumulators being adapted to calculate the plurality of health risk factors and generate a group health risk factor. The system of further comprises a group activity selector 138 coupled to the long-term health risk factor generator and adapted to select a group activity from a plurality of group activities based upon the group health risk factor. The system further comprises a group insurance calculator 142 coupled to the long-term health risk factor generator and adapted to determine at least one of an insurance reserve and an insurance premium associated with the group health risk factor based upon the group health risk factor.

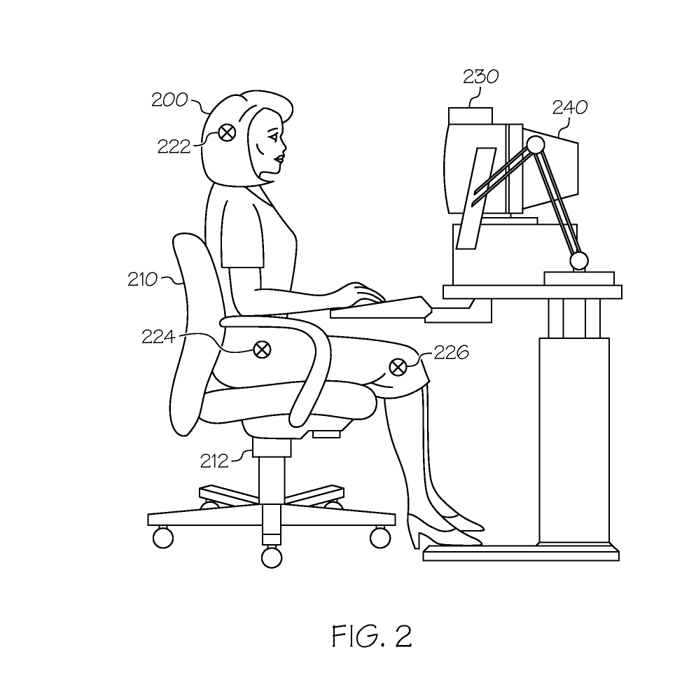

FIG. 2 illustrates an example of an individual posture monitor for monitoring a posture of a subject person. The individual posture monitor of FIG. 2 provides a more detailed description of individual posture monitors 110 and/or 120 of FIG. 1. Subject person 200 is shown seated in a chair 210. The chair includes a sensor 212 that determines the amount of weight experienced by the seat of the chair. The weight signal may be analyzed by the seated posture analyzer 112 and if the weight exceeds a certain amount, then a seated posture may be determined and a seated signal generated. The chair may be a “smart chair” and may include a number of sensors including sensor 212, arm rest sensors, back rest sensors, neck-rest sensors, tilt sensors and chair translational and/or rotational motion sensors in order to more accurately determine a seated posture or other postures of the subject person. Numerous other posture determining systems and devices based upon sensors incorporated into devices, such as furniture, including office chairs and desks, are known to those familiar with the art and all such sensor systems for determining posture are considered to be within the scope of this description.

The individual posture monitor may also include sensors 222, 224 and 226 mounted on the body or clothing of the subject person 200. For example, sensor 222 may include a Bluetooth headset worn in the ear of the subject person. Sensor 222 may be in communication with a cell phone worn by the subject person 200. The cell phone may also act as a second sensor 224 worn at the waste of the subject person. A third sensor 226 may be included in the clothing of the subject person and located at the knee of the subject person. If it is determined that the three sensors for an angle indicative of a seated posture, then the seated posture analyzer may determine the subject person has a seated posture. The angle indicative of a seated posture may be an angle exceeding forty five degrees or approaching ninety degrees. If sensors 222-226 included Zigbee transceivers or a transceiver capable of determining time of flight or distance between transceivers, then the seated angle may be readily determined. Numerous other posture determining systems and devices based on body and or clothing worn sensors are known to those familiar with the art and all body and/or clothing worn sensor systems for determining posture are considered to be within the scope of this description.

The individual posture monitor of FIG. 2 may also include a camera 230 or other optical system for determining the posture of a subject person. The camera may be used to determine that the subject person is seated in a chair and thus generate a seated signal. In another example, an optical system such as the Kinect system provided by Microsoft may be used to determine the posture of the subject person. Based upon the determine posture, it may be determined if the subject person has a seated posture and a seated signal is generated in response. The camera system or optical system may include a plurality of cameras in a plurality of locations. Locations may include cameras mounted in meeting rooms, hallways, building entrances and exits, parking lots, athletic facilities or portable cameras such as those included in cell phones, tablets, laptop computers, head mounted devices, or any other camera or optical system. Other such camera or other optical systems for determining individual posture are considered to be within the scope of this description.

The individual posture monitor of FIG. 2 may also include a computer 240 with which the subject person 200 interfaces. The computer may be used to determine whether or not the subject person has a seated posture based on the context of information available to the computer. For example, if the office furniture is configured to be used while the subject person is in the seated position, and if the subject person is using the computer, then it may be determined that the subject person 200 is seated while using the computer 240. A seated signal may be generated in response. Furthermore, if the computer, or network including the computer, includes a calendar application including information associated with activities of the subject person, then the calendar application may be analyzed to determine if the subject person is seated during the activity. For example, if the subject person is scheduled to attend a meeting in a location set up for a seated meeting, then a seated signal may be generated based on the meeting. However, if the location was set up for a stand-up meeting or other non-seated activity then the seated signal would not be generated based upon the calendar application. Thus, a computer system's calendar application may be one of a plurality of devices for generating at least one of the plurality of seated signals for at least one of the plurality of subject persons. Other applications on the computer system may be used for generating seated signals based on the application indicating whether or not the subject person is seated.

Posture monitors 110, 120 may be coupled to a network for sending generated posture signals. The coupling may be a wired or wireless connection. The seated posture analyzer may be included in the posture monitor and may be implemented by a process or program instructions operating on a microprocessor, microcomputer, microcontroller, or processor including a non-transitory computer program comprising a computer readable storage medium having computer readable program code embodied therewith. The computer readable program code may be configured to implement at least some of the processes described herein. Furthermore, the processes may be at least partially implemented by application specific integrated circuit. The network may connect the posture monitor server to the posture monitors, which may implement the remainder of the processes of FIG. 1. The processes of FIG. 1 may further be distributed amongst the various components of a server based network system and additional components may be included in the posture monitor such as the seated posture accumulator and the individual characteristic database. Multiple different partitions of the processes and components of FIG. 1 are possible while remaining within the scope of this description.

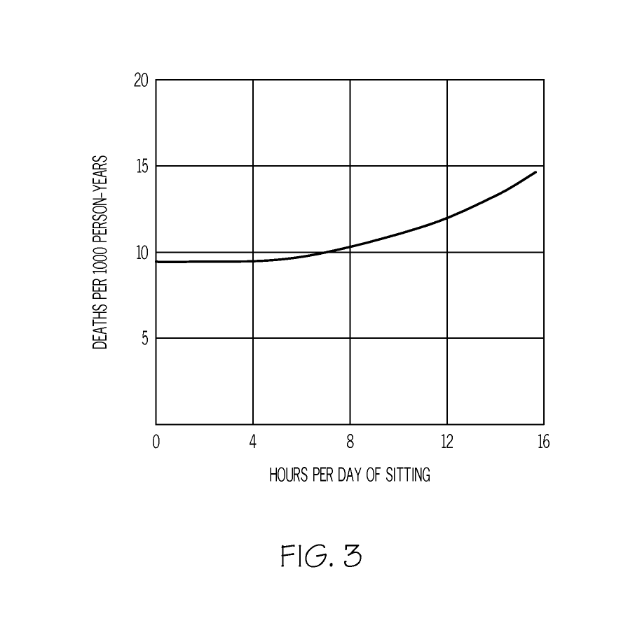

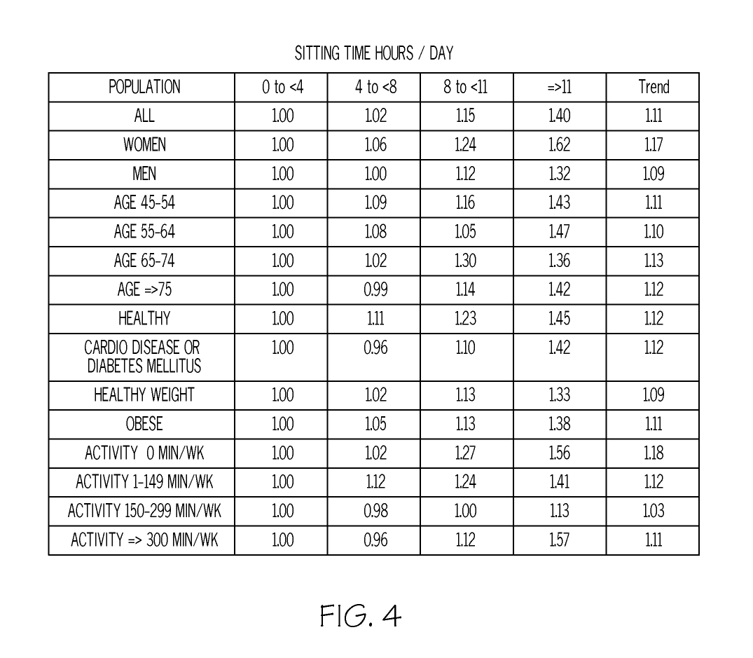

FIG. 3 illustrates an example graph showing deaths per thousand person-years verses hours per day of sitting. The graph is based on analysis of data from 222, 497 Australian adults. It shows that the mortality rate of the population increases with hours per day of sitting. The curve of FIG. 4 is for the entire population. The shape of the curve may change based upon various individual characteristics, but the curve substantially consistently shows and increase in mortality rate with hours per day of sitting. Thus, it has been determined that an increase in sitting time increases the all-cause mortality risk of the population.

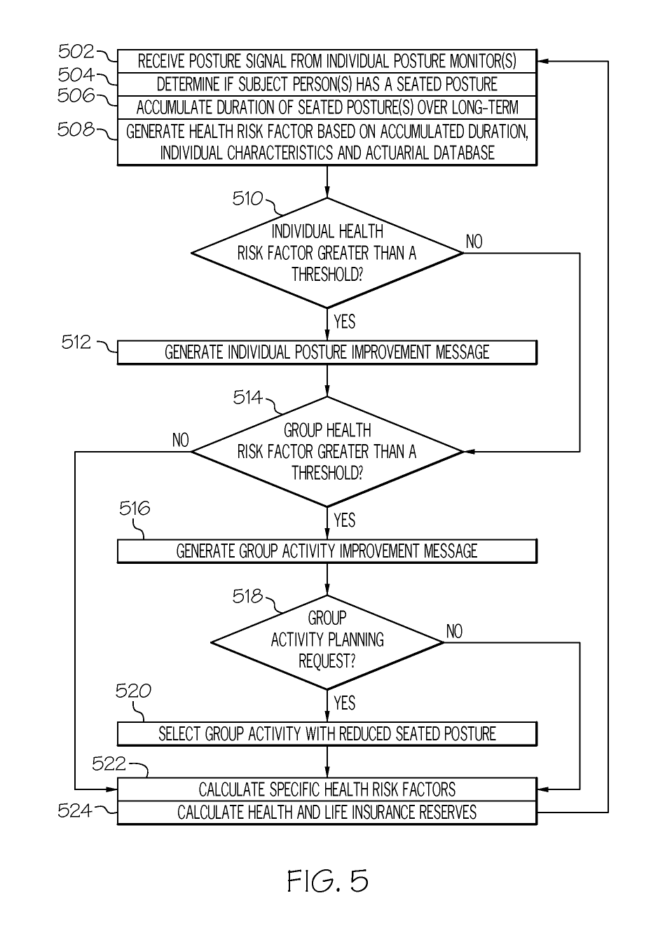

FIG. 4 illustrates a table showing an association between sitting and all-cause mortality among Australian adult forty five years or older. The table shows results from the aforementioned publication, “Sitting Time and All-Cause Mortality Risk in 222 497 Australian Adults” by Hidde P. van der Ploeg, PhD; Tien Chey, MAppStats; Rosemary J. Korda, PhD; Emily Banks, MBBS, PhD; Adrian Bauman, MBBS, PhD ARCH INTERN MED/VOL 172 (NO. 6), Mar. 26, 2012, 498. The first column shows individual characteristics that may be included in individual characteristic databases 114 and 124. Additional individual variable or characteristics may also be included. For example, individual characteristics may include sex, age, education level, marital status, location residence, body mass index, smoking status, health status, serological analysis, genetic analysis, and physical activity of the subject person. Other individual characteristics may also be included and are intended to be within the scope of this description. The second through fifth columns show the change in mortality rate based increases sitting hours per day. The sixth column shows the trend of the prior columns. The actuarial database 132 may be at least in part based upon the information shown in FIG. 4. The table of FIG. 5 shows a relative mortality rate based upon sitting time and other individual characteristics. Other tables showing other health risks based on sitting time may also be generated, such other health risks include cardiovascular disease mortality and a type two diabetes mellitus. All health risks based on sitting time and/or corresponding actuarial databases are intended to be within the scope of this description.

FIG. 5 illustrates a flow diagram example of a process for determining health risk factor based upon sitting time and associated responses. Step 502 receives a posture signal from individual posture monitors. Step 504 determines if the subject person or persons has a seated posture, and if so accumulates the duration of the seated postures. The accumulated postures are accumulated over a long term and available for analysis by the health risk factor generator. The long term may be seven days or thirty days or longer. Step 508 generates a health risk factor based upon accumulated durations, individual characteristics and the actuarial database. Step 510 then determines if an individual health risk factor exceeds a threshold. The threshold could be based on a predetermined mortality risk value of ten deaths per thousand person-years and the calculated individual health risk factor exceeds the threshold, then the individual posture improvement message of step 512 would be generated. The threshold for the individual could be tailored to the individual based upon the individual characteristics and the actuarial database. The individual posture improvement message of step 512 may encourage activities that reduce the individual's sitting time or other posture or health improvement messages, such messages may include sitting straighter, changing positions of arms or hands, or even increasing levels of fidgeting to improve components of the health of the person. Step 514 then determines if the group health risk factor exceeds a threshold. If so, step 516 generates a group activity improvement message, which may be a message to the entire group or to a manager of a group responsible for activities of the group. The message may further be in the form of a report or a signal to another device or process indicating the risk factors. In a different example, the report or signal may be generated independent of the threshold and provide information on the calculated risk. Step 518 then determines if a group activity planning request has been made. Such a request may be to arrange a meeting of the group of designated persons using a computer based meeting facilitator application. Since the threshold had been exceeded in step 514, step 520 selects a group activity with a reduced seating time posture, such as a meeting forum configured for a standing up meeting. It should be appreciated that a different group of persons may not result in the group health risk factor exceeding the threshold and thus a standing up meeting forum may not be recommended at step 520, even though both groups may be members of a larger group, such as a large employer, and even though both groups may have one or more member persons in common Thus, the selected activity of step 520 may be tailored to the specific group of persons involved in the activity. Step 522 then calculates specific health risk factors as shown in 140 of FIG. 1. Step 524 then calculates health and life insurance reserves and premiums associated with the group.

It should be appreciated that the system, method and processes of the description analyze the long-term effects of prolonged seating and recommend responses. The accumulated seated posture signal for subject persons may be analyzed every seven days or on a week-by-week basis, every thirty days or on a month-by-month basis or over longer periods. Group analytics may also be based on the long-term effects of prolonged seating of identified groups. For example, the long-term health risk factor may be generated on a month-by-month basis in order to determine changes in the long-term health risk factor. Such changes may be used to determine impact of individual and group posture messages and the group activities selected by the group activity selector. Furthermore, the group insurance calculator can adjust insurance premiums and reserves established for the group. In one example, the group activity selector may result in reduced seating times, which results in a reduced long-term health risk factor, which may allow for a reduction in health and/or life insurance premiums as well as a reduction in insurance reserves maintained by an employer or an insurance company associated with the group, thereby allowing for more efficient use of monetary capital. This also allows for adjusting an insurance premium associated with the plurality of subject persons based upon the group health risk factor and further in response to the participation in the group activity.

The respective implementations of the present disclosure can be carried out in any appropriate mode, including hardware, software, firmware or combination thereof. Alternatively, it is possible to at least partially carry out the implementation of the present disclosure as computer software executed on one or more data processors and/or a digital signal processor. The components and modules or processes of the implementation of the present disclosure can be implemented physically, functionally and logically in any suitable manner. Indeed, the function can be realized in a single member or in a plurality of members, or as a part of other functional members. Thus, it is possible to implement the implementation of the present disclosure in a single member or distribute it physically and functionally between different members and a processor.

Computer program code for carrying out operations for aspects of the present disclosure may be written in any combination of one or more programming languages, including an object oriented programming language such as Java, Smalltalk, C++ or the like and conventional procedural programming languages, such as the “C” programming language or similar programming languages. The program code may execute entirely on the user's computer, partly on the user's computer, or entirely or partly on the user's electronic handheld devices, as a stand-alone software package, partly on the user's computer or handheld device, and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Aspects of the present disclosure are described herein with reference to flowchart illustrations flow diagrams and/or block diagrams of methods, apparatus (systems) and computer program products according to implementations of the disclosure. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the computer or other programmable data processing apparatus, create means for implementing the functions/acts specified in the blocks of the flowchart illustrations and/or block diagrams.

These computer program instructions may also be stored in a computer readable medium that can direct a computer or other programmable data processing apparatus to function in a particular manner, such that the instructions stored in the computer readable medium produce an article of manufacture including instruction means which implement the functions/acts specified in the blocks of the flowchart illustrations and/or block diagrams.

The computer program instructions may also be loaded onto a computer or other programmable data processing apparatus to cause a series of operational steps to be performed on the computer or other programmable data processing apparatus to produce a computer implemented process such that the instructions which execute on the computer or other programmable apparatus provide processes for implementing the functions/acts specified in the blocks of the flowchart illustrations and/or block diagrams.

The present disclosure is described by use of detailed illustration of the implementations of the present disclosure, and these implementations are provided as examples and do not intend to limit the scope of the present disclosure. Although these implementations are described in the present disclosure, modifications and variations on these implementations will be apparent to those of ordinary skill in the art. Therefore, the above illustration of the exemplary implementations does not confine or restrict the present disclosure. Other changes, substitutions and modifications are also possible, without departing from the scope of the description and the appended claims.

What is claimed is:

1. A method comprising:
receiving a seated signal from a device monitoring a posture of a subject person;
accumulating the seated signal over a duration; and
generating a health risk factor based upon the accumulating, wherein at least one of the receiving, the accumulating and the generating above is performed by a computer.

2. The method according to claim 1 wherein the seated signal is indicative of the subject person being in a seated position and the health risk factor does not specify modification of the posture of the subject person.

3. The method according to claim 2 wherein the health risk factor is associated with a mortality of the subject person.

4. The method according to claim 2 wherein the health risk factor is associated with at least one of a cardiovascular disease mortality and a type two diabetes mellitus of the subject person.

5. The method according to claim 1 wherein the generating generates the health risk factor further based upon at least one additional individual variable associated with the subject person, the at least one additional individual variable including at least one of sex, age, education level, marital status, location residence, body mass index, smoking status, health status, serological analysis, genetic analysis, and physical activity of the subject person.

6. The method according to claim 1 wherein
the duration for accumulating the seated signal is greater than seven days, and
the health risk factor is generated after the duration.

7. The method according to claim 1 wherein
the duration for accumulating the seated signal is greater than thirty days, and
the health risk factor is generated after the duration.

8. The method according to claim 1 wherein the device is one of a smart chair and a context aware posture recognition system.

9. The method according to claim 1 further comprising
generating a long-term posture modification signal for the subject person based upon the health risk factor wherein the posture modification signal modifies an amount of time the subject person is seated over a period of time greater than or equal to seven days.

10. The method according to claim 1 further comprising
generating a long-term posture modification signal for the subject person based upon the health risk factor wherein the posture modification signal encourages at least one of fidgeting and standing.

11. The method according to claim 1 wherein
the receiving further receives a plurality of seated signals from a plurality of devices monitoring postures of a plurality of subject persons,
the accumulating further accumulates the plurality of seated signals, and
the generating further generates a group health risk factor for the plurality of subject persons based upon the accumulating.

12. The method according to claim 11 further comprising
selecting a group activity from a plurality of group activities for the plurality of subject persons based upon the group health risk factor.

13. The method according to claim 12 wherein the plurality of group activities includes a seated meeting forum and an unseated meeting forum and the method selects between the seated meeting forum and the unseated meeting forum based upon the group health risk factor.

14. The method according to claim 12 further comprising
adjusting an insurance premium associated with the plurality of subject persons based upon the group health risk factor and a participation in the group activity.

15. The method according to claim 11 wherein the plurality of devices includes a computer system having a calendar application for at least one of the plurality of subject persons and the method further comprises generating the at least one of the plurality of seated signals based upon the calendar application.

16. A system comprising:
a plurality of posture monitors adapted to analyze a posture of each of a plurality of subject persons and generate a plurality of seated signals based on the analysis;
a plurality of posture accumulators adapted to accumulate the plurality of seated signals;
a plurality of individual characteristic databases including individual characteristic information on each of the plurality of subject persons;
an actuarial database having information relating each of the plurality of posture accumulators to of each of the individual characteristic databases and adapted to be used in generating a plurality of health risk factors; and
a long-term health risk factor generator coupled to the actuarial database, the plurality of individual characteristic databases and the plurality of posture accumulators, the long-term health risk factor generator adapted to calculate the plurality of health risk factors and generate a group health risk factor signal.

17. The system of claim 16 further comprising a group activity selector coupled to the long-term health risk factor generator and adapted to select a group activity from a plurality of group activities based upon the group health risk factor signal.

18. The system of claim 16 further comprising a group insurance calculator coupled to the long-term health risk factor generator and adapted to determine at least one of an insurance reserve and an insurance premium based upon the group health risk factor signal.

19. A non-transitory computer program comprising a computer readable storage medium having computer readable program code embodied therewith, the computer readable program code configured to:
receiving a seated signal from a device monitoring a posture of a subject person;
accumulating the seated signal over a duration; and
generating a health risk factor based upon the accumulating.

20. The non-transitory computer program according to claim 19 wherein
the receiving further receives a plurality of seated signals from a plurality of devices monitoring postures of a plurality of subject persons;
the accumulating further accumulates the plurality of seated signals; and
the generating further generates a group health risk factor for the plurality of subject persons based on the accumulating.
